(19) United States
(12) Patent Application Publication
(10) Pub. No.: US 2022/0016193 A1
(43) Pub. Date: Jan. 20, 2022

(54) ANTIBACTERIAL AND ANTIVIRUS COMPOSITION COMPRISING Extract of CANNABIS SATIVA L.

(71) Applicant: FAMENITY CO., LTD., Uiwang-si (KR)

(72) Inventor: Ji Won LEE, Gwacheon-si (KR)

(21) Appl. No.: 17/375,715

(22) Filed: Jul. 14, 2021

(30) Foreign Application Priority Data
Jul. 20, 2020 (KR) 10-2020-0089803

(51) Int. Cl. A61K 36/185; A61K 9/00; A61P 31/16; A61P 31/04

(52) U.S. Cl. CPC A61K 36/185; A61K 9/0056; A61K 9/0014; A61P 31/16; A61P 31/04; A61K 2236/331; A61K 2236/51; A61K 2236/15; A61K 2236/13; A61K 2236/17; A61K 2236/37; A61K 2236/39; A61K 2236/53; A61K 2236/333

(57) ABSTRACT

The present disclosure relates to an antibacterial and antiviral composition containing an extract of Cannabis sativa L. as an active ingredient. More specifically, the present disclosure provides an antibacterial and antiviral composition which contains an extract of the natural product Cannabis sativa L., and thus may exhibit antibacterial and antiviral activities, and when taken or administered, has no side effects and does not cause bacterial or viral resistance.

### CROSS-REFERENCE TO RELATED APPLICATION

This application claims priority from and the benefit of Korean Patent Application No. 10-2020-0089803, filed on Jul. 20, 2020, which is hereby incorporated by reference for all purposes as if set forth herein.

### BACKGROUND

### Field

The present disclosure relates to an antibacterial and antiviral composition containing an extract of Cannabis sativa L. as an active ingredient. More specifically, the present disclosure provides an antibacterial and antiviral composition which contains an extract of the natural product Cannabis sativa L., and thus may exhibit antibacterial and antiviral activities, and when taken or administered, has no side effects and does not cause bacterial or viral resistance.

### Discussion of the Background

It is known that about 480 species among about 3,200 species of plants that grow naturally in Korea may be used for edible purposes. Edible plants are referred to as wild vegetables in a broad sense. In fact, among plants eatable by humans, plants with good palatability and high food value are used as wild vegetables. Wild vegetables are hardy plants that have been used from ancient times mainly when our ancestors suffered from food shortages. In addition, wild vegetables are rich in vitamins, minerals, nutrients such as chlorophyll, and plant fiber, and particularly, have high food value due to their unique flavor and taste. Currently, about 100 species of wild vegetables are known. Since wild vegetables contain a lot of various alkaloids, tannins, saponins, glycosides, and flavonoids, they are known to have a very high possibility of exhibiting special physiological functions. In Korea, the food value of edible plants that grow naturally in Korea has been re-recognized, and the demand therefor has increased every year, and studies have been actively conducted to identify physiologically active substances from foods, herbal medicines, and folk medicines.

In recent years, it has been found that wild vegetables have a preventive effect on chronic degenerative diseases resulting from nutritional imbalance, and also have high anti-obesity effects, heavy metal detoxification effects, anti-mutagenic effects, anti-cancer and antioxidant effects, antibacterial effects and genotoxic inhibitory ability, and thus necessity of research and development on the physiological functions of various wild vegetables has been emphasized.

In particular, food poisoning, one of diseases that frequently occur in the summer, is mainly caused by ingesting food contaminated by bacteria, including Staphylococcus aureus, Bacillus cereus, Salmonella, E. coli, Listeria, etc., which are major food poisoning bacteria. Infection with food poisoning bacteria may be naturally cured when it is mild, but antibiotic prescription is required when the symptoms of the infection are severe.

In general, in order to treat severe infections with Staphylococcus aureus, vancomycin, which is one of the most powerful antibiotics among antibiotics developed to date, is used in some cases. However, vancomycin has a disadvantage that it causes the resistance of Staphylococcus aureus thereto when used for a long period of time. For example, vancomycin resistant Staphylococcus aureus (VRSA) showing strong resistance to vancomycin was found in 1996, and in addition, a considerable number of multi-drug resistant bacteria such as methicillin-resistant Staphylococcus aureus (MRSA) and carbapenem-resistant Acinetobacter baumanii (CRAB) were found. In order to kill these antibiotic-resistant bacteria, there has been an increasing demand for a new alternative that may replace synthetic antibiotics.

The problem with the emergence of such super-bacteria is that the time taken for bacteria to acquire resistance to antibiotics is incomparably faster than the time taken to develop new antibiotics. Resistant bacteria rapidly spread to animals and plants in various ways, so that consumers potentially carry resistant bacteria by ingesting foods contaminated with the resistant bacteria.

Thus, in consideration of the emergence of these multi-drug resistant bacteria having resistance to the antibiotics and the side effects of the antibiotics, there has been increasing interest in natural plant-derived antibacterial substances that are easily available and may be used for a long time because of their relatively few side effects such as bacterial resistance.

Meanwhile, the term “virus” is Latin for poisonous substance and is a group of infectious pathogenic particles that pass through bacterial filter paper (0.22 μm). Viruses can be classified into bacteriophage, plant virus, and animal virus according to the type of host cell, and can also be classified into DNA virus and RNA virus according to the type of nucleic acid. In recent years, various viral diseases such as swine flu, AI and foot-and-mouth disease have caused great social problems, and accordingly, effective measures against viral diseases have attracted a great deal of social attention.

Currently, the best way to prevent viral diseases is vaccination, but in the case of diseases caused by viruses, the effectiveness of vaccines has become an important issue due to according to the generation of a large number of virus serotypes (subtypes). The development and distribution of inhibitors for viral prevention that can overcome this problem of vaccines is an important matter. To this end, particularly, the discovery and development of preventive agents that increases the immunity of individual animals by stimulating the innate immune system in vivo, which is an early defense system against viruses, can be an important method of developing antiviral agents.

Representative antiviral agents that inhibit the multiplication of influenza virus include amantadine and rimantadine. However, it has been found that these two antiviral agents are effective only against influenza A virus, but are not effective against influenza B virus having no M2 protein. In addition, it has been found that amantadine and rimantadine have a disadvantage that the emergence of a mutant virus, the ion channel function of the influenza virus M2 protein of which is not affected by the use of these drugs, occurs very easily. In order to overcome this disadvantage, zanamivir and oseltamivir were developed, which are antiviral agents effective against all 16 types of influenza A virus and influenza B virus. However, zanamivir has a disadvantage in that it should be administered by inhalation and intravenously. Oseltamivir may be administered orally, but the emergence of oseltamivir-resistant viruses has recently been reported, and oseltamivir has a disadvantage in that it has side effects such as vomiting and dizziness when administered orally.

In addition, the main and best way to control vesicular stomatitis virus is preventive biosecurity and the eradication of susceptible domestic animals within the virus-hit area, like foot-and-mouth disease, because complete treatment of vesicular stomatitis virus disease is impossible.

In addition, vaccines against Newcastle disease virus are broadly divided into live vaccines and inactivated vaccines. The representative Newcastle disease live vaccines B1 strain and La Sota strain (including colon strain), which have been most widely used, are known to cause vaccination reactions, and a multivalent mixed inactivated oil vaccine, a Newcastle disease inactivated vaccine which is used worldwide, can prevent three or more diseases at the same time by a single vaccination. However, in laying hen farms, cases of damage caused by Newcastle disease due to decreased immunity have increased.

In addition, among the RNA viruses, there are relatively small viruses. These viruses are called picornaviruses by combining the term “pico” meaning small and the term “RNA”, and these viruses belong to the Picornaviridae family. Enteroviruses belonging to the Picornaviridae family include about 70 serotypes that cause various clinical symptoms such as aseptic meningitis, hand-foot-and-mouth disease, herpes stomatitis, dilated myocarditis, and acute hemorrhagic conjunctivitis, and are classified into poliovirus, Coxsackie virus, echo virus, and other enteroviruses. Enteroviruses are about 20 to 30 nm in diameter and have a single-stranded RNA as a gene. Enteroviruses mostly infect the respiratory organs, central nervous system and digestive organs of vertebrates, but infection with enteroviruses shows no obvious symptoms in many cases. Coxsackie virus (CXV) is a human enterovirus belonging to the Picornavirus family, and is broadly divided into type A and type B.

In addition, high-risk enteroviruses and their mutant viruses (enterovirus type 71 (EV-71) and Coxsackie virus A24 variants) have been newly found and prevalent in all parts of the world, and thus it is needed to establish an international monitoring system for monitoring these viruses early. Since enteroviruses including Coxsackie virus cause various clinical symptoms by infecting the respiratory organs, central nervous system and digestive organs of vertebrates, it is urgently required to prepare measures against enterovirus infection at the national level. However, the types and serotypes of viruses are very diverse, and hence effective commercial vaccines or therapeutic agents have not been developed.

Under these circumstances, in Korea and foreign countries, many efforts have recently been made to overcome the disadvantages of existing antiviral agents. As one of these efforts, studies on the antiviral effects of herbal extracts and plant extracts have been conducted in Korea, but still remain insufficient. Therefore, there is a need to develop compositions containing, as active ingredients, herbal extracts that may overcome the disadvantages of existing antiviral agents and exhibit excellent antibacterial effects and antiviral activities while having little toxicity and few side effects.

### PRIOR ART DOCUMENTS

### Patent Documents

(Patent Document 0001) KR 10-1471710 B1

### SUMMARY

An object of the present disclosure is to provide an antibacterial and antiviral composition containing an extract of Cannabis sativa L. as an active ingredient.

Another object of the present disclosure is to provide an antibacterial and antiviral composition which contains an extract of the natural product Cannabis sativa L., and thus exhibits antibacterial and antiviral activities and, at the same time, causes few side effects when taken or administered.

To achieve the above objects, an antibacterial and antiviral composition according to one embodiment of the present disclosure contains an extract of Cannabis sativa L. as an active ingredient.

The composition exhibits an antibacterial effect by effectively removing bacteria selected from the group consisting of Streptococcus mutans, Staphylococcus aureus, Candida albicans, Bacillus subtilis, Escherichia coli and Staphylococcus aureus.

The composition has an antiviral effect against A/H1N1/WSN influenza virus.

The extract is obtained by extraction with an extraction solvent selected from the group consisting of water, a C₁ to C₆ lower alcohol, and a mixture thereof.

A food composition according to another embodiment of the present disclosure is produced using the antibacterial and antiviral composition containing an extract of Cannabis sativa L. as an active ingredient.

A skin external preparation according to still another embodiment of the present disclosure is produced using the antibacterial and antiviral composition containing an extract of Cannabis sativa L. as an active ingredient.

A medicament according to yet another embodiment of the present disclosure is produced using the antibacterial and antiviral composition containing an extract of Cannabis sativa L. as an active ingredient.

Hereinafter, the present disclosure will be described in more detail.

As used herein, the term “extract” not only means a crude extract that is commonly used in the art as described above, but also includes, in a broad sense, a fraction obtained by fractionating the extract. That is, the term “extract” includes not only an extract obtained using the above-described extraction solvent, but also one obtained by additionally applying a purification process to the extract. For example, the term “extract” as used herein also include a fraction obtained by passing the extract through an ultrafiltration membrane having a certain molecular weight cut-off value, and fractions obtained by additionally performing various purification processes, such as separation by various chromatography systems (manufactured for separation according to size, charge, hydrophobicity or affinity).

An antibacterial and antiviral composition according to one embodiment of the present disclosure contains an extract of Cannabis sativa L. as an active ingredient.

Cannabis sativa L. is an annual plant belonging to the genus Cannabis of the family Cannabaceae, and is divided into a total of three subspecies: C. sativa subsp. sativa, C. sativa subsp. indica, and C. sativa subsp. ruderalis. It is known that Cannabis sativa L. has been cultivated widely over tropical and temperate regions, particularly Central Asia, for about 12,000 years in the past, when legal regulations on Cannabis sativa L. were not applied, and Cannabis sativa L. was used for edible purposes in China around 6,000 BC. There is a record that fiber obtained from Cannabis sativa L. was used around 4,000 BC, and there is a Chinese herbal book describing that Cannabis sativa L. was first used in 2,727 BC for medical purposes. However, since the 20th century, cultivation and handling of Cannabis sativa L. have been strictly regulated in most countries as well as in Korea due to adverse effects thereof, such as hallucinations.

However, in foreign countries, the importance of the excellent pharmacological action of Cannabis sativa L. has been gradually recognized, and there is an increasing movement to permit the use of Cannabis sativa L. for therapeutic purposes.

In Korea, there is a lack of studies on Cannabis sativa L. due to strict regulations on Cannabis sativa L., and there are also almost no studies, particularly on efficacy tests and toxicity related problems of Cannabis sativa L. for medical purposes.

Cannabis sativa L. has been used in various ways in the past depending on parts thereof. Specifically, it is known that the leaf of Cannabis sativa L. has the effect of killing roundworms, and that, when hair is washed with water obtained by boiling the leaf of Cannabis sativa L., the hair grows long and becomes abundant. In addition, the leaf of Cannabis sativa L. was used against asthma or old cough or roundworms, or as an analgesic, anesthetic or diuretic agent. There is a record that the root of Cannabis sativa L. was used for the treatment of difficult delivery and “placenta not coming out”, the removal of extravasated blood, and the treatment of urolithiasis, and was taken as a water decoction. There is a record that the shell of Cannabis sativa L. was used for the treatment of bruises and fever-type intestinal pain, and the flower of Cannabis sativa L. was used for paralysis symptoms and itching. The flower spike of Cannabis sativa L. was used for difficulty delivery, constipation, gout, manic depressive psychosis, insomnia, and the like.

As far as is known, about 400 compounds have been found in Cannabis sativa L., and most of them are cannabinoids, terpenes, and phenolic compounds. Among them, cannabinoids are known as representative active ingredients of Cannabis sativa L. About 90 kinds of cannabinoids have been identified to date, and a number of ingredients found only in Cannabis sativa L are also known. Cannabinol (CBN) was isolated from Cannabis sativa L. in 1899, but it was later found that the cannabinol was not a single compound. Since cannabidiol (CBD) and tetrahydrocannabinol (THC), which are pure compounds, were isolated from Cannabis sativa L. in the 1930s, studies on the components of Cannabis sativa L. have been more actively conducted.

Efforts to develop drugs using specific components of Cannabis sativa L. have also been continued, and among these specific components, THC and CBD, which are major compounds of Cannabis sativa L., have attracted the most attention for therapeutic purposes. Some studies indicated that CBD has no THC is psychotropic action and is effective in reducing pain and controlling epileptic seizures.

In addition, more than 100 terpene-based compounds that play a role in the flavor and taste of Cannabis sativa L. were also found in Cannabis sativa L., and are present as various monoterpenoids and sesquiterpenoids. Terpenes have been found to be related to various pharmacological actions such as anti-inflammatory action, but studies on terpene compounds extracted from Cannabis sativa L. are still insufficient compared to THC.

In addition, terpenes are known to exhibit better effects when acting together with cannabinoids such as CBD and THC, and may improve the uptake of cannabinoids, overcome the bacterial defense mechanism and minimize side effects.

The composition exhibits an antibacterial effect by effectively removing bacteria selected from the group consisting of Streptococcus mutans, Staphylococcus aureus, Candida albicans, Bacillus subtilis, Escherichia coli and Staphylococcus aureus.

There is a normal flora inside and outside the human body, which rather promotes the body's metabolism. Our body has many defense mechanisms, and hence even when bacteria invade the body, the bacteria are killed in the stomach and small intestine, and immune cells in the blood prevent infection. Thus, the bacteria do not immediately lead to disease. However, these bacteria can also become a problem for people with weak immunity. In particular, these bacteria can be fatal to people suffering from immune diseases, such as AIDS patients or cancer patients. In addition, when bacteria invade newborns or the elderly with weak immunity, or persons who take overdoses of antibiotics, the bacteria may immediately cause disease.

The Streptococcus mutans is a bacterial species present in the oral cavity of a normal person, is related to the early stage of enamel damage, and is a major cause of tooth decay. Sugar is decomposed to synthesize insoluble dextran, and the dextran aggregates oral bacteria, including mutans, to form plaques, and plaque metabolites (acids) cause enamel demineralization and tooth decay.

The Staphylococcus aureus is a Gram-positive coccal bacterium, is an opportunistic infectious bacterium that causes problems in hospitals, and has recently been designated as representative super-bacteria such as MRSA or VRSA. It is a pathogen that causes inflammation and necrosis due to various toxins and various infection sites, and has penicillin resistance even though it is a Gram-positive bacterium sensitive to penicillin. It is found in the nose or skin in 20 to 30% of adults, but does not cause a problem in most cases. However, as the defense system collapses due to skin damage or injury, infection with Staphylococcus aureus occurs. Staphylococcus aureus is associated with impetigo, cellulitis, scalded skin syndrome, mastitis, bacteremia, staphylococcal sepsis, staphylococcal pneumonia, endocarditis, etc., and food poisoning from Staphylococcus aureus causes nausea, vomiting, diarrhea, and bowel disease causing dehydration.

The Candida albicans is a representative fungus commonly observed in the oral cavity and gastrointestinal tract, and is found in 50% of the oral cavities of normal persons and in 90% of the feces. In most cases, C. albicans causes non-specific lesions that have no clinical significance, but may cause various diseases depending on the patient's immunity. C. albicans can be found anywhere in the gastrointestinal tract, but most frequently causes lesions in the throat and esophagus, and these lesions are mostly asymptomatic. C. albicans rarely causes fungal infections in the stomach and duodenum, but these fungal infections are mostly secondary lesions that metastasized to and proliferated in benign or malignant ulcers, and are known to have little clinical significance.

The Bacillus subtilis mainly lives in soil and is a Gram-positive bacterium belonging to the genus Bacillus. Bacillus subtilis is an aerobic, rod-shaped or round bacterium, and has various sizes and lengths, and the shapes of both ends thereof are not uniform. It may have flagella or spores. It may be mobile by having 1 to 3 flagella at one end of the cell or a number of flagella around the cell, or may be a bacillus having spores at one end or the center of the cell. The spores of the bacterium are portions in which the bacterial cells are in a resting state, and the spores do not lead to life phenomena, but have strong resistance to the influence of the external environment. Thus, the spores can survive for a long time even under bad living conditions, and germinate in a suitable environment and become unique bacterial cells. That is, the spores of the bacterium are the same as bacterial seeds. In addition, Bacillus subtilis is associated with a microorganism used for Cheonggukjang, a Korean traditional fermented food, and is also used as various microbial agents. Bacillus subtilis is known to be associated with conjunctivitis and iritis.

The Escherichia coli is one of the bacteria that reside in the intestines of humans or mammals, and refers, in a narrow sense, to a bacterium belonging to the genus Escherichia of the family Enterobactericeae, and in a broad sense, a coliform group including three E. coli strains, Citrobacter, Klebsiella, and Enterobacter. The Escherichia coli in the narrow sense is a

Gram-negative bacillus of 0.5 to 0.7 μm in width and 2 to 3 μm in length and is peritrichous, but some E. coli strains have no flagellum. The Escherichia coli develops well in normal culture media, and forms smooth, opaque and white colonies. E. coli decomposes glucose, lactose and mannitol (sometimes also decomposes sugar and salicin), and generates acids and gases. As pathogenic Escherichia coli (a kind of bacteria that mainly causes diarrhea in infants), 0-55, 0-111, 0-124, etc. have been reported. In general, Escherichia coli is a bacterium that resides in the mammalian intestinal tract, and has a meaningful existence value for the host. That is, the decomposition ability of E. co/i described above is exerted in the intestinal tract, and is also related to the production of vitamins. In addition, the coliform group often serves as a measure of fecal contamination, which is a standard for sanitary inspection in drinking water or food, and furthermore, in beaches or public baths. This is because the presence of fecal contamination indicates the presence of food poisoning bacteria Salmonella and pathogenic Escherichia co/i in addition to dysentery bacillus or typhoid bacterium which is the infectious pathogenic bacterium of the digestive system, or raises this doubt. However, the coliform group itself is not harmful, and is used as a pollution indicator because it does not proliferate in nature other than the intestinal tract. In addition, Escherichia co/i is known to be associated with urethral infection, cystitis, renal infection, urethral infection, gastroenteritis, peritonitis, appendicitis, pneumonia, meningitis, bone or joint infection, etc.

The Staphylococcus aureus is an important pathogenic bacterium which is commonly isolated from patients, and causes infections such as bacteremia, osteomyelitis and meningitis in addition to respiratory infections, and the most common infection with Staphylococcus aureus is a skin infection. Staphylococcus aureus is a skin flora that worsens acne that occurred. Acne, which is one of the representative skin inflammatory diseases, refers to an inflammatory disease of the pilosebaceous gland, hair follicles and surrounding tissues, which is caused by the action of hormones, excessive sebum secretion, and bacterial infection.

Methods for treatment of acne include topical drug application therapy, oral drug administration therapy, and surgical therapy such as laser surgery and dermabrasion, which have been recently performed. The topical application therapy is a method of either using an antibacterial agent such as azelaic acid or salicylic acid, which has antibacterial activity against acne-causing bacteria, or using benzoyl peroxide as an external ointment type for the purpose of dissolving comedones, etc. However, this method has difficulty in formulation and also has difficulty in commercialization due to related laws and regulations. The oral drug administration therapy is a method of using retinoids, corticosteroids, etc. for the purpose of suppressing the function of sebaceous glands and destructing the hair follicle wall, and has a problem in that when these agents are administered in excessive amounts, they cause side effects such as skin redness, skin hypersensitivity reaction, and dry skin.

The present disclosure has been made in order to solve the above-described problems, and may provide an antibacterial composition which contains an extract of Cannabis sativa L. as an active ingredient, and thus exhibits an antibacterial effect and, when taken or administered, has no side effects and does not cause bacterial resistance.

The composition has an antiviral effect against A/H1N1/WSN influenza virus.

H1N1 influenza virus has an official name of influenza A virus subtype H1N1, and is the most common type among influenza viruses that affect humans. H1N1 influenza virus may affect and cause infection in pigs and birds as well as humans, and specific examples thereof include A/PR/8(H1N1), A/WSN/33(H1N1), A/Bervig-Mission/1/18(rvH1N1), A/Singapore/6/86(H1N1), and the like.

Swine flu A (H1N1) virus is a virus that caused the 2009 flu pandemic, and examples thereof include A/California/04/09, A/California/7/2009, and the like. The swine flu A (H1N1) virus can cause flu, cold, sore throat, bronchitis, and pneumonia.

Conventional drugs that are used for the treatment of influenza include oseltamivir (trade name: Tamiflu), zanamivir (trade name: Relenza), en Peramivir, en Amantadine, and the like. Oseltamivir, called Tamiflu, is currently mainly used in the treatment of influenza A virus H1N1. Tamiflu is the only avian influenza (AI) therapeutic agent which is exclusively produced in the world. Tamiflu is an antiviral agent that exhibits a therapeutic effect by blocking the enzyme function that multiplies viruses, and it is highly effective only when it is taken within 48 hours after the onset of symptoms. The main therapeutic effects of Tamiflu include a decrease in worsening of influenza symptoms, a decrease in occurrence of secondary complications such as bronchitis or pneumonia, and a decrease in the incubation period for influenza. Tamiflu is also used as a therapeutic agent for influenza A and B. Zanamivir is commercially available under the trade name Relenza. Zanamivir acts as a neuraminidase inhibitor and is used for the treatment of influenza A and B.

However, oseltamivir has a side effect of severe vomiting symptoms, and zanamivir has a high antiviral effect, but has the disadvantages of low bioavailability and rapid excretion from the kidney. Anti-influenza therapeutic agents developed to date mostly show side effects.

For this reason, the present disclosure may provide a composition which contains an extract of Cannabis sativa L., and thus exhibits an antiviral effect against A/H1N1/WSN influenza virus and at the same time, is effective in the prevention and treatment of influenza without the above-described side effects.

Preferably, the antibacterial and antiviral composition containing an extract of Cannabis sativa L. as an active ingredient may further contain an extract of Vicia heptajuga Nakai, an extract of Calluna vulgaris and an extract of Nigella sativa.

The Vicia heptajuga Nakai is a perennial plant growing in mountains. The leaves thereof are alternate and are paripinnately compound leaves, each composed of 4 to 6 pairs of lobules. The leaves have traces of tendrils at the ends thereof, and the bottom thereof is dull. The ends of the leaves are long pointed, and the edges thereof are flat. The flowers thereof bloom in red purple in June through July. The flower stalk having a length of 2 to 4 cm comes out from the axil of the leaf, and the flowers are clustered in a racemose inflorescence.

The Calluna vulgaris is the only species of the genus Calluna, a monotypic genus of the Rhododendron family, and is a perennial shrub and grows well in acidic soil in sunny places or slightly shady places. Calluna vulgaris is native to a region ranging from Siberia through Turkey and Europe to North Africa and Macaronesia. Calluna vulgaris is an evergreen small shrub having a height of about 60 cm and has strong cold resistance. Branches of Calluna vulgaris densely grow while spreading laterally. The leaves of Calluna vulgaris are scaly, about 2 to 3 mm in size, adhere densely to the branches, and face each other. The flowers thereof are small bell-shaped, bloom in June through September, adhere densely to a part ranging from the middle to the upper part of the branch, and are applied as various garden varieties having various colors, including white and pink.

The Nigella sativa is an annual or biennial dicotyledonous plant belonging to the family Ranunculaceae of the order Ranunculales. Nigella sativa is an annual plant that grows to a height of about 50 to 80 cm. The leaves thereof are split pinnately 3 to 4 times to form the shape of cosmos leaves and are glossy. The flowers of Nigella sativa bloom in June through July, are light purple in color, and each hang at the end of each branch. The fruits thereof are plump and ball-shaped, and the seeds thereof are small and black and ripen in August through September. Nigella sativa is native to southern Europe. The strawberry-scented seeds of Nigella sativa are widely distributed in India, Egypt, Greece, and Turkey, and have a spicy nutmeg taste. The roasted and dried seeds of Nigella sativa are used to add flavor to curry, vegetable and bean dishes.

When the natural extracts are used in combination, the antibacterial and antiviral effects thereof are increased due to the synergistic action between the natural extracts.

In addition, as the extract of Vicia heptajuga Nakai, the extract of Calluna vulgaris and the extract of Nigella sativa are additionally contained, it is possible to provide a composition having excellent palatability by neutralizing the unique taste and flavor of the extract of Cannabis sativa L.

Preferably, the composition of the present disclosure may contain, based on 100 parts by weight of the extract of Cannabis sativa L., 20 to 40 parts by weight of the extract of Vicia heptajuga Nakai, 20 to 40 parts by weight of the extract of Calluna vulgaris, and 20 to 40 parts by weight of the extract of Nigella sativa.

When the extracts are used in combination in amounts within the above-described ranges, it is possible to provide a composition having excellent palatability while exhibiting excellent antibacterial and antiviral effects.

The extract is obtained by extraction with an extraction solvent selected from the group consisting of water, a C₁ to C₆ lower alcohol, and a mixture thereof.

Specifically, the extract of Cannabis sativa L. as a natural extract may be obtained by a method including steps of: crushing a natural product to obtain a sample; leaching the sample with an organic solvent; drying the leached sample; re-leaching the dried sample with an organic solvent; drying the re-leached sample; leaching the dried sample with water; and leaching.

The natural extract obtained by extraction with the organic solvent may be further subjected to a fractionation step using an organic solvent.

The extraction solvent may be used in an amount equal to 2 to 50 times, more specifically 2 to 20 times, the weight of the sample. For leaching and extraction, the sample may be left to stand in the extraction solvent for 1 to 72 hours, more specifically 24 to 48 hours.

The extract may be prepared in a powder state by additional processes such as reduced pressure distillation and freeze drying or spray drying, and is obtained by an extraction method selected from the group consisting of a solvent extraction method, an ultrasonic extraction method, a reflux extraction method, a leaching method, a fermentation method, and a processing method.

The ultrasonic extraction method is performed by extraction using water or a 50 to 100% alcohol having 1 to 6 carbon atoms as an extraction solvent at 30 to 50° C. for 0.5 to 2.5 hours. Specifically, the ultrasonic extraction method includes performing extraction using water or a 70 to 80% alcohol having 1 to 6 carbon atoms as an extraction solvent at 40 to 50° C. for 1 to 2.5 hours.

The reflux extraction method is performed by refluxing 10 to 30 g of the crushed natural product in 100 mL of water or a 50 to 100% alcohol having 1 to 6 carbon atoms for 1 to 3 hours. More specifically, the reflux extraction method is performed by refluxing 10 to 20 g of the crushed natural product in 100 mL of water or a 70 to 90% alcohol having 1 to 4 carbon atoms for 1 to 2 hours.

The leaching method is performed using water or a 50 to 100% alcohol having 1 to 6 carbon atoms as an extraction solvent at 15 to 30° C. for 24 to 72 hours. More specifically, the leaching method is performed using water or a 70 to 80% alcohol having 1 to 6 carbon atoms as an extraction solvent at 20 to 25° C. for 30 to 54 hours.

After extraction, the extract may be fractionated sequentially using fresh fractionation solvents. The fractionation solvent that is used for fractionation of the extract is any one or more selected from the group consisting of water, hexane, butanol, ethyl acetic acid, ethyl acetate, methylene chloride, and mixtures thereof. Preferably, the fractionation solvent is ethyl acetate or methylene chloride.

The antibacterial and antiviral composition containing an extract of Cannabis sativa L. as an active ingredient according to the present disclosure may be used in various applications. For example, the extract of Cannabis sativa L. may be used as an active ingredient for a health food for immune enhancement, a health food such as a tea or beverage for preventing or treating food poisoning or promoting digestion, a health food for preventing or treating influenza virus infection diseases such as cold, cosmetic products such as bath products, cosmetics, detergents or hair products, an antibacterial agent against Escherichia coli, Staphylococcus, Bacillus, Salmonella, Listeria, Acinetobacter, vancomycin-resistant Staphylococcus aureus (VRSA), methicillin-resistant Staphylococcus aureus (MRSA) or carbapenem-resistant Acinetobacter baumanii (CRAB), an antiviral agent against A/H1N1/WSN influenza virus, or an agent for preventing or treating food poisoning or cold.

A functional food according to another embodiment of the present disclosure is produced using the antibacterial and antiviral composition containing an extract of Cannabis sativa L. as an active ingredient.

As used herein, the term “functional food” refers to foods produced and processed using functional raw materials or ingredients beneficial to human health pursuant to Health Functional Foods Act No. 6727, and the term “functionality” means controlling nutrients for the structure or functions of the human body or providing beneficial effects to health purposes, such as physiological effects.

A skin external preparation according to still another embodiment of the present disclosure is produced using the antibacterial and antiviral composition containing an extract of Cannabis sativa L. as an active ingredient.

A medicament according to yet another embodiment of the present disclosure is produced using the antibacterial and antiviral composition containing an extract of Cannabis sativa L. as an active ingredient.

The dosage form of the medicament of the present disclosure may be preferred form selected depending on the method of use thereof, and specific examples of the dosage form include granules, powders, syrups, liquids, suspensions, decoctions, infusions, tablets, suppositories, injections, spirits, capsules, pills, and soft or hard gelatin capsules.

In addition, if necessary, the medicament of the present disclosure may further contain an excipient, a filler, an extender, a binder, a disintegrant, a lubricant, a preservative, an antioxidant, an isotonic agent, a buffer, a film-forming agent, a sweetening agent, a solubilizing agent, a base agent, a dispersing agent, a wetting agent, a suspending agent, a stabilizer, a colorant, a fragrance, etc. which are commonly used in the art.

In the manufacture of the medicament, the content of the antibacterial or antiviral composition according to the present disclosure may vary depending on the form of the medicament, and the dosage thereof may be easily adjusted by those skilled in the art depending on the type of subject to be treated, the route of administration, the subject's weight, sex, age, and the severity of the disease.

### BRIEF DESCRIPTION OF THE DRAWINGS

FIG. 1 shows the results of measuring the cell viability of A549 cells treated with a composition according to one embodiment of the present disclosure before infection (pre-treatment), after infection (post-treatment), and before and after infection (pre-post-treatment) with A/H1N1/WSN influenza virus.

### DETAILED DESCRIPTION OF THE ILLUSTRATED EMBODIMENTS

Hereinafter, examples of the present disclosure will be described in detail so that those of ordinary skill in the art can easily carry out the present disclosure. However, the present disclosure may be embodied in a variety of different forms and is not limited to the examples described herein.

### Production Example 1: Production of Extracts

1. Production of Cannabis sativa L. Extract

Cannabis sativa L. including leaves and flowers was washed clean with running water, and then completely dried naturally. The dried Cannabis sativa L. was crushed with a mixer and then prepared into powder. The powder sample was added to a 50% ethanol as an extraction solvent at a ratio of 1:10 (w:v), and then completely immersed in the solvent. Next, the powder sample was extracted under reflux at 80° C. three times for 3 hours each. The extract was filtered through Whatman No. 2 filter paper. The filtrate was concentrated under reduced pressure at 60° C., thereby producing a Cannabis sativa L. extract (CE).

2. Production of Other Natural Extracts

A Vicia heptajuga Nakai extract (VE), a Calluna vulgaris extract (RE) and a Nigella sativa extract (NE) were produced according to the same method as the method for producing the Cannabis sativa L. extract (CE).

3. Production of Extract mixtures

The Cannabis sativa L. extract (CE), the Vicia heptajuga Nakai extract (VE), the Calluna vulgaris extract (RE) and the Nigella sativa extract (NE) were mixed together as shown in Table 1 below to obtain extract mixtures.

TABLE 1

| | AM1 | AM2 | AM3 | AM4 | AM5 | AM6 |
|---|---|---|---|---|---|---|
| CE | 100 | 100 | 100 | 100 | 100 | 100 |
| VE | — | 10 | 20 | 30 | 40 | 50 |
| RE | — | 10 | 20 | 30 | 40 | 50 |
| NE | — | 10 | 50 | 30 | 40 | 50 |

(unit: parts by weight)

### Test Example 1: Cytotoxicity Test

To test the toxicity of each of the Cannabis sativa L. extract (CE) (AM1) and the extract mixtures (AM2 to AM6), differences in toxicity and side effects caused by administration of the extract mixtures in repeated-dose toxicity tests for rats were examined.

6-week-old male and female SD rats were divided into a plurality of groups, each consisting of 10 rats (5 male rats and 5 female rats), and each of the extract mixtures AM1 to AM6 was administered to the rats. Each of the extract mixtures was dissolved in a 0.5% methylene chloride (MC) solution and then administered orally once at the same time in the morning every day. This administration was repeated for 13 weeks. Each of the extract mixtures was administered once a day at a daily dose of 3.75 mg/kg to 5 mg/kg. Thereafter, mortality, general symptoms, weight changes, and feed and water intakes were observed.

As a result, no death occurred within the test period. In view of the above test results, it was confirmed that the extract mixtures AM1 to AM6 had no toxicity problem.

### Test Example 2: Antibacterial Effect

In order to measure the antibacterial activities of the Cannabis sativa L. extract (CE) and extract mixtures AM1 to AM6 produced in Production Example 1, a modification of the agar diffusion method (Piddock, L. J. V, Techniques used for the determination of antimicrobial resistance and sensitivity in bacteria, J. Appl. Bacteriol, 68, p. 307, 1990) using a paper disk was performed as follows.

First, for antibacterial activity analysis, Escherichia coli (KCTC7039), Bacillus subtilis (KCTC1021), Candida albicans (KCTC7270), Streptococcus mutans (KCTC3065) and Staphylococcus aureus (KCTC1621) were obtained from the Korea Collection for Type Cultures (KCTC), the Korea Research Institute of Bioscience and Biotechnology, the Korea Advanced Institute of Science and Technology, and used in the following experiment. The bacterial strains were used after subculture (Candida albicans: 24° C., Bacillus subtilis: 30° C., Streptococcus mutans: 37° C., Staphylococcus aureus: 37° C., and Escherichia coli: 37° C.).

One loop of each of the prepared strains was taken and inoculated in 5 ml of saline, and 1 ml of each of the active solutions was inoculated into a 4 to 5-mm-thick medium (Candida albicans—YM Agar (271210. BD), Bacillus subtilis—Nutrient Agar (213000 BD), Streptococcus mutans—Brain Heart (241830 BD), Staphylococcus aureus—Tryptic Soy Agar (236950 BD), Escherichia coli—Nutrient Agar (213000 BD); incubator: EYELA Multi-Thermo incubator MTI-202), and then spread evenly with a bent glass rod. Then, a 6.0-mm sterile filter paper disc (Whatman AA Disc) was placed on each of the media, and a sample solution consisting of a 1:5:4 (v/v/v) mixture of each of the extract mixtures AM1 to AM6, 95% ethanol and distilled water was injected at concentrations of 500 μg/ml, 1000 μg/ml, 1500 μg/ml, 2000 μg/ml and 2500 μg/ml.

The solvent of each injected extract mixture was volatilized, and incubation was performed at 37° C. for 48 hours. Then, the diameter of the inhibition zone formed around each paper disc was measured. In addition, as a control, butylated hydroxyanisole (BHA) (SIGMA), which is currently widely used as a synthetic antioxidant, was used, and measurement for the control was performed in the same manner as described above

The results of the measurement are shown in Table 2 below.

TABLE 2

| | Inhibition zone (mm) | | | | | | | |
|---|---|---|---|---|---|---|---|---|
| | Concentration | | | | | | | |
| | (mg/ml) | AM1, CH | AM2 | AM3 | AM4 | AM5 | AM6 | BHA |
| Bacillus subtilis | 2.5 | 7.6 | 8.2 | 10.4 | 20.6 | 24.8 | 19.3 | 16.6 |
| Escherichia coli | 2.5 | 7.0 | 10.0 | 17.6 | 36.0 | 38.6 | 23.4 | 18.4 |
| Candida albicans | 2.5 | 3.0 | 3.0 | 5.0 | 7.6 | 8.2 | 7.0 | 16.0 |
| Staphylococcus aureus | 2.5 | 3.0 | 4.0 | 4.0 | 6.8 | 6.2 | 5.0 | 16.0 |
| Streptococcus mutans | 2.5 | 2.0 | 3.0 | 5.0 | 8.4 | 15.4 | 6.8 | 3.5 |

(unit: mm)

As shown in Table 2 above, BNA used as the control exhibited some degree of antibacterial effects, but exhibited little antibacterial effect against S. mutans.

It was confirmed that the extract mixtures AM1 to AM6 of the present disclosure exhibited overall antibacterial effects, and had little antibacterial effect against C. albicans and S. aureus, but AM4 and AM5 exhibited excellent antibacterial effects against B. subtillis and E. coli.

### Test Example 3: Antiviral Effect

1. Cell Culture

MDCK Cell Line

MDCK cells (Madin-Darby canine kidney cell line, No: 10034), which are epithelial-like cells established from the kidneys of Cocker Spaniel species, were cultured in DMEM containing 10% FBS (Fetal Bovine Serum) and 1% penicillin/streptomycin in a cell culture chamber at 37° C. under 5% CO₂.

A549 Cell Line

A549 cells (human lung adenocarcinoma epithelial cell line, ATCC, No. CCL-185), which are epithelial cells established from human lung cancer cells, were cultured in RPMI 1640 (Roswell Park Memorial Institute 1640) containing 10% FBS, 1% penicillin/streptomycin and 1% non-essential amino acids in a cell culture chamber at 37° C. under 5% CO₂.

2. Culture of Influenza Virus

Construction of Reporter Influenza Virus

A plasmid DNA set required for construction of influenza virus (A/H1N1/WSN) was obtained from UCLA (USA) and transfected into 293T cells and MDCK cells together with a reporter plasmid expressing green fluorescent protein (GFP), thereby constructing reporter influenza virus expressing green fluorescence. MDCK cells were infected with the constructed influenza virus, and the viral infectivity of the cells was determined based on the expression level of GFP.

Multiplication Culture of Influenza Virus in Fertilized Eggs

Specific pathogen-free (SPF) fertilized eggs were grown at 37° C. for 10 days, and then a dilution of the above-constructed A/H1N1/WSN influenza virus was injected into the allantoic cavity, and the virus was multiplied at 37° C. for 2 days, followed by collection of the allantoic fluid (containing the virus).

3. Cell Viability Test

Since infection with influenza virus shows cytotoxicity, treatment with an antiviral agent may exhibit the effect of reducing cytotoxicity. Thus, before infection (pre-treatment), after infection (post-treatment) and before and after infection (pre-post-treatment) (MOI=0.5) with A/H1N1/WSN influenza virus, A549 cells were treated with each of AM1 to AM6, and after 3 days, the viability of the cells was measured by MTT assay.

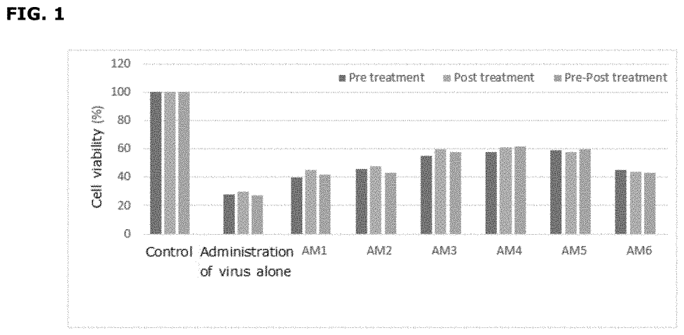

As a result, as shown in FIG. 1, it was confirmed that the cytotoxicity of the cells treated with each of AM1 to AM6 according to the present disclosure decreased compared to that of the cells to which the virus was administered alone.

### Test Example 4: Palatability Test

1. Sensory Evaluation Test

Tea beverages were prepared by diluting each of the Cannabis sativa L. extract (CE) and the extract mixtures AM2 to AM6. Each of the tea beverages were tasted by 10 panelists, and the taste and flavor thereof were scored on a 10-point scale (1 to 10). The average values of the scores (any fraction of 0.5 or more is rounded up to the next higher whole number) are shown in Table 3 below. In the scores in Table 3 below, a higher score indicates higher palatability.

TABLE 3

| | AM1 | AM2 | AM3 | AM4 | AM5 | AM6 |
|---|---|---|---|---|---|---|
| Taste | 6.0 | 6.0 | 6.5 | 7.0 | 7.5 | 6.0 |
| Flavor | 6.0 | 6.5 | 6.5 | 7.0 | 7.5 | 7.0 |
| Overall palatability | 6.0 | 6.0 | 7.0 | 7.0 | 7.5 | 6.5 |
| (average) | | | | | | |

(unit: score)

Referring to Table 3 above, it can be seen that, in the case of AM1 composed of the Cannabis sativa L. extract (CE) alone, the palatability was lowered due to the unique taste and flavor of the Cannabis sativa L. extract, and in the case of the mixtures AM2 to AM6, the palatability increased while the unique taste and flavor of the Cannabis sativa L. extract were neutralized by the other extracts.

In particular, it was confirmed that, in the case of AM3 to AM5, the palatability greatly increased while the flavor was highly evaluated.

Therefore, each of the extract mixtures AM3 to AM5 according to the present disclosure may provide a functional food having an excellent antibacterial and antiviral effect while having higher flavor and taste palatability.

As described above, the present disclosure may provide an antibacterial and antiviral composition which contains an extract of Cannabis sativa L. as an active ingredient, and thus exhibits antibacterial and antiviral activities, and when taken or administered, has no side effects and does not cause bacterial or viral resistance.

Although the preferred embodiments of the present disclosure have been described in detail above, the scope of the present disclosure is not limited thereto, and various modified and improved forms made by those skilled in the art on the basis of the basic concept of the present disclosure defined in the appended claims also fall within the scope of the present disclosure.

What is claimed is:

1. An antibacterial and antiviral composition containing an extract of Cannabis sativa L. as an active ingredient.

2. The antibacterial and antiviral composition of claim 1, wherein the composition exhibits an antibacterial effect by effectively removing bacteria selected from the group consisting of Streptococcus mutans, Staphylococcus aureus, Candida albicans, Bacillus subtilis, Escherichia coli and Staphylococcus aureus.

3. The antibacterial and antiviral composition of claim 1, wherein the composition has an antiviral effect against A/H1N1/WSN influenza virus.

4. The antibacterial and antiviral composition of claim 1, wherein the extract is obtained by extraction with an extraction solvent selected from the group consisting of water, a C₁ to C₆ lower alcohol, and a mixture thereof.

5. A functional food comprising the antibacterial and antiviral composition according to claim 1.

6. A skin external preparation comprising the antibacterial and antiviral composition according to claim 1.

7. A medicament comprising the antibacterial and antiviral composition according to claim 1.
